(19) United States
(12) Patent Application Publication
(10) Pub. No.: US 2018/0261317 A1
(43) Pub. Date: Sep. 13, 2018

(54) BALANCED DIET PORTION TRACKING SYSTEM

(71) Applicant: Darren V. Michie, Stevenson, WA (US)

(72) Inventor: Darren V. Michie, Stevenson, WA (US)

(21) Appl. No.: 15/916,264

(22) Filed: Mar. 8, 2018

Related U.S. Application Data
(60) Provisional application No. 62/468,652, filed on Mar. 8, 2017.

(51) Int. Cl. G16H 20/60; G09B 29/00; G09B 19/00

(52) U.S. Cl. CPC G16H 20/60; G09B 19/0092; G09B 29/001

(57) ABSTRACT

A system for tracking food balance and portion control includes a food-board, a plurality of first board-markers, and a plurality of second board-markers. The system for tracking food portion control is useful for providing a system for easily tracking portion control for a customizable balance diet. The system provides means for weigh control and proper portion eating.

### CROSS REFERENCE TO RELATED APPLICATION

The present application is related to and claims priority to U.S. Provisional Patent Application No. 62/468,652 filed Mar. 8, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of diet systems and more specifically relates to a system for tracking balanced diet portions.

### 2. Description of Related Art

For years, maintaining a physically fit and healthy body has been the goal of an increasing number of individuals. As a major part of this goal, these individuals have employed various diets, either to lose weight, to maintain one's present weight, or to assure that only well-balanced meals are consumed. While existing weight-loss systems have a variety of useful features, such systems are typically cumbersome to use due in part to the complexities of adequate data entry. The difficulties in various data entry steps in such systems are likely to discourage consistent use over a long period of time for many users. Also, individuals in good health who may have very specific health and fitness goals such as bodybuilders, runners, martial arts enthusiasts and others may utilize such a system to help attain their fitness and health goals by monitoring and tracking exercise, behavioral and nutrition parameters. Thus, a suitable solution is desired.

U.S. Pat. No. 5,683,251 to Kathleen Logan relates to a system and method for tracking dietary consumption. The described system and method for tracking dietary consumption includes a board divided into a plurality of regions and a plurality of tokens adapted to stick to the board. The tokens comprise at least: a) a plurality of first tokens, each of the first tokens corresponding to a single portion of a food or beverage item selected from a first specific food group; and b) a plurality of second tokens, each of the second tokens corresponding to a single portion of a food or beverage item selected from a second specific food group. The regions on the board comprise at least: a) a first region, where the first region is labelled as corresponding to the first specific food group; and b) a second region, where the second region is (1) labelled as corresponding to the second specific food group.

### BRIEF SUMMARY OF THE INVENTION

In view of the foregoing disadvantages inherent in the known diet systems art, the present disclosure provides a novel balanced diet portion tracking system. The general purpose of the present disclosure, which will be described subsequently in greater detail, is to provide a system for easily tracking portion control for a customizable balance diet.

A system is disclosed herein. The system includes a food-board which may include a food-table being defined by at least two portion-columns and at least six food-rows (five rows for vegans or seven rows with milk and bean food groups separated; and one water row), the at least six food-rows each may have at least one food-group indicia. Two different food groups may be in the same row if their calorie count per portion is the same. For example, milk and beans having the same calorie count per portion.

Further, the system may include a plurality of row-cells, and the at least one food-group indicia may be different on each of the at least six food-rows. Further, each of the plurality of row-cells may include at least one food-portion indicia corresponding to the at least one food-group indicia on each of the at least six food-rows (and in some embodiments, one water row). A plurality of first board-markers may be also be included. The plurality of first board-markers may be configured for attachment to the food-board and include a first-format. Similarly, a plurality of second board-markers may be provided and configured for attachment to the food-board. The plurality of second board-markers may include a second-format, and the second-format may be readily distinguishable from the first-format. Further board-markers may be included for more than two users and each set of board-markers may be readily distinguishable from the other.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a balanced diet portion tracking system, constructed and operative according to the teachings of the present disclosure.

FIG. 1 is a front perspective view of the system during an ‘in-use’ condition, according to an embodiment of the disclosure.

FIG. 2 is a front perspective view of the system of FIG. 1, according to an embodiment of the present disclosure.

FIG. 3 is a front perspective view of the system of FIG. 1, according to an embodiment of the present disclosure.

FIG. 4 is a front perspective view of the system of FIG. 1, according to an embodiment of the present disclosure.

FIG. 5 is a front perspective view of the system of FIG. 1, according to an embodiment of the present disclosure.

FIG. 6 is a front perspective view of the system of FIG. 1, according to an embodiment of the present disclosure.

FIG. 7 is a front perspective view of the system of FIG. 1, according to an embodiment of the present disclosure.

FIG. 8 is a front perspective view of the system of FIG. 1, according to an embodiment of the present disclosure.

FIG. 9 is a front perspective view of the system of FIG. 1, according to an embodiment of the present disclosure.

FIG. 10 is a front perspective view of the system of FIG. 1, according to an embodiment of the present disclosure.

FIG. 11 is a front perspective view of the system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a diet system and more particularly to a balanced diet portion tracking system as used to improve the system for tracking balanced diet portions.

Generally, the balanced diet portion control tracking system may be a simple way to make sure balanced diets are adhered to every day and to help prevent health complications and low energy levels. The balanced diet portion tracking system may be made up of 6 food group rows and 1 water row. Each row may be made up of boxes and each box may represent a portion of food for that food groups row.

Further, the system may include 14 marker piece magnets for each user. 7 of them may include one color which for example may be red, and 7 of them may include another color which for example may be green. The red magnets may be used one in each row for the stopping point of each food group. Similarly, the green magnets may be used, one in each row, to move along as a user eat from one meal to the next, never passing the red stop magnets. The user may ask their doctor where to put the red stop magnet markers, and what would be a perfect balanced diet for the user.

A larger balanced diet portion tracking system may include food portions written on them and on a separate page with the instructions. A smaller personal balanced diet portion tracking system may include the food portions written in with the instructions only. “Balanced Diet Portion Tracking System” may be manufactured in different sizes and arrangements and calorie limits. Examples systems for vegetarians may exclude milk from its row and exclude protein entirely.

All “Balanced Diet Portion Tracking Systems” may be provided with instructions, and the instructions may include four different diet options. The four different diet options may include: one “1200 Calorie Weight Loss Diet”; one average “Dr. Recommend Balanced Diet”; one “Build Bulk Weight Lifters Diet”; and one “Get Lean Weight Lifters Diet”. These diets may be included in with the instructions to help people learn where to put their red stop markers on the “Balanced Diet Portion Tracking System”. Other systems may only cover a limited amount of calories, for small, medium and large people.

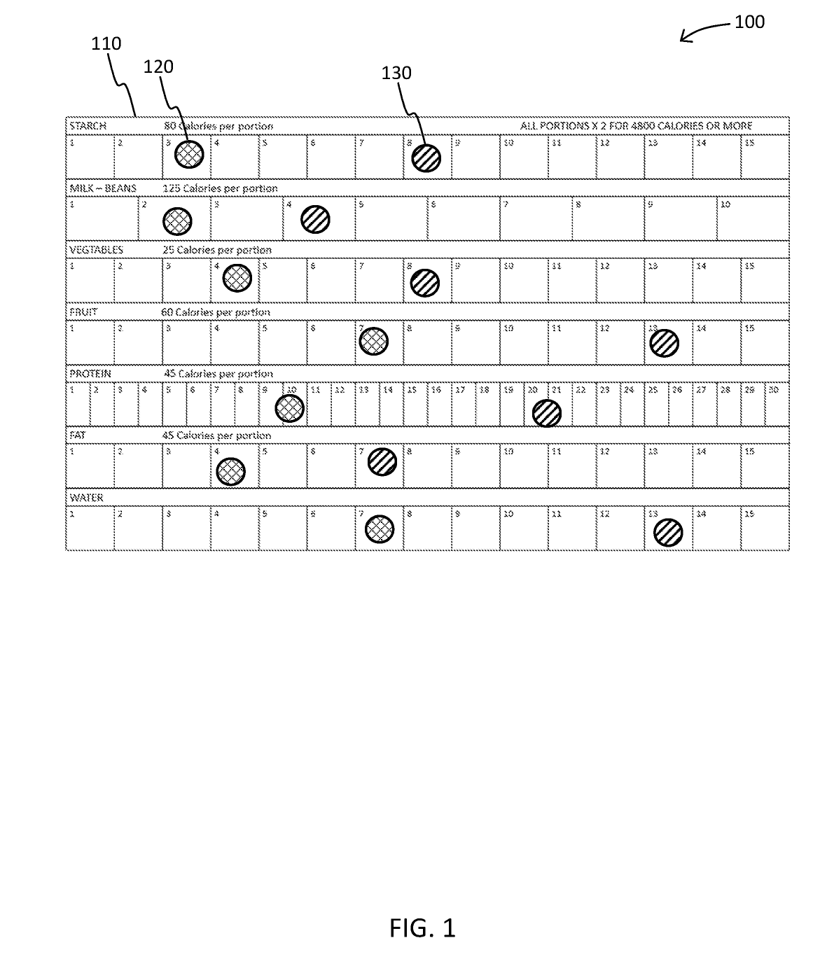

Referring now more specifically to the drawings by numerals of reference, there is shown in FIGS. 1-11, various views of a system 100.

FIG. 1 shows a system 100 during an ‘in-use’ condition 150, according to an embodiment of the present disclosure. As illustrated, the system 100 may include a food-board 110, a plurality of first board-markers 120, and a plurality of second board-markers 130. The system 100 may be used for tracking food portion control.

In a preferred embodiment, the food-board 110, the first board-markers 120, and the second board-markers 130 may be magnetic, and the first board-markers 120 and the second board-markers 130 may be configured for magnetic-attachment to the food-board 110. However, in other embodiments, other attachment-methods may be used. For example, hook and loop fastener, snap fasteners, and the like.

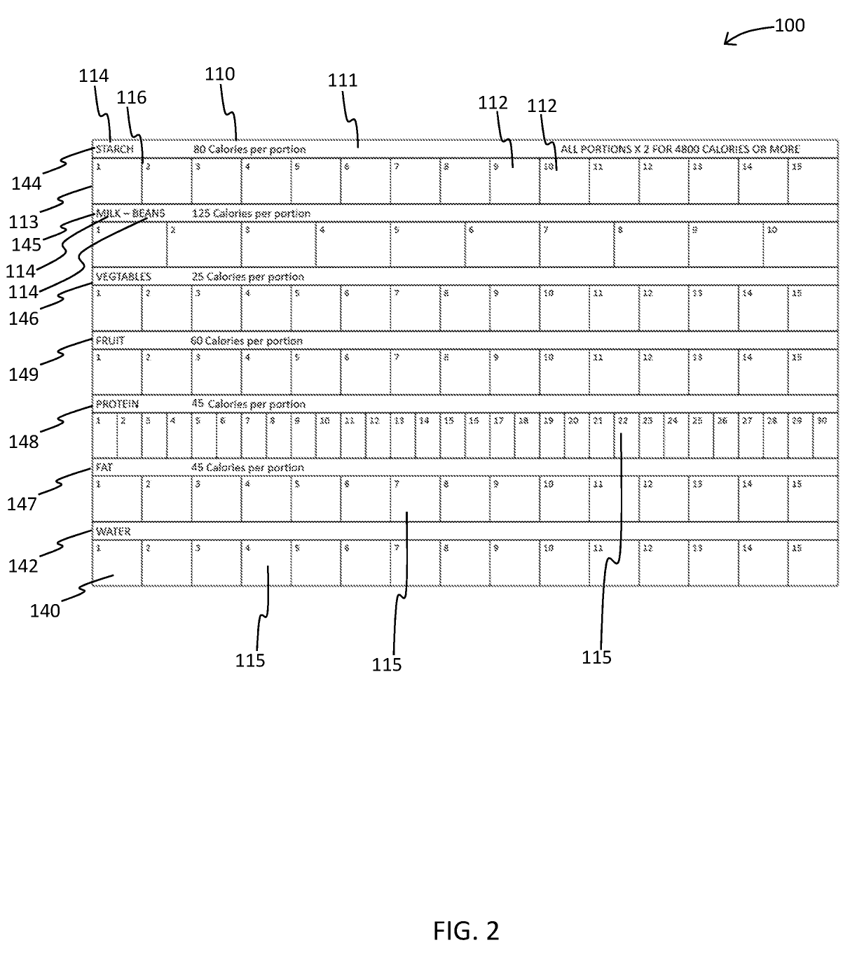

Referring now to FIG. 2 showing a front perspective view of the system 100 of FIG. 1, according to an embodiment of the present disclosure. As shown, the food-board 110 may include a food-table 111 being defined by at least two portion-columns 112 and at least six food-rows 113. The at least six food-rows 113 may each have at least one food-group indicia 114 and a plurality of row-cells 115, and the at least one food-group indicia 114 may be different on each of the at least six food-rows 113.

As illustrated, the at least one food-group indicia 114 may be starch 144, milk-beans 145, vegetables 146, fats 147, proteins 148, and fruits 149. In a preferred embodiment, one of the at least six food-rows 113 may be starch 144, another may be milk-beans 145, another may be vegetables 146, another may be fats 147, another may be proteins 148 and another may be fruits 149. Further, as shown, each of the plurality of row-cells 115 may include at least one food-portion indicia 116 corresponding to the at least one food-group indicia 114 on each of the at least six food-rows 113.

Further, as illustrated, the system 100 may include at least one liquid-row 140 including at least one liquid-group indicia 142. In a preferred embodiment, the liquid-group indicia 142 may represent water.

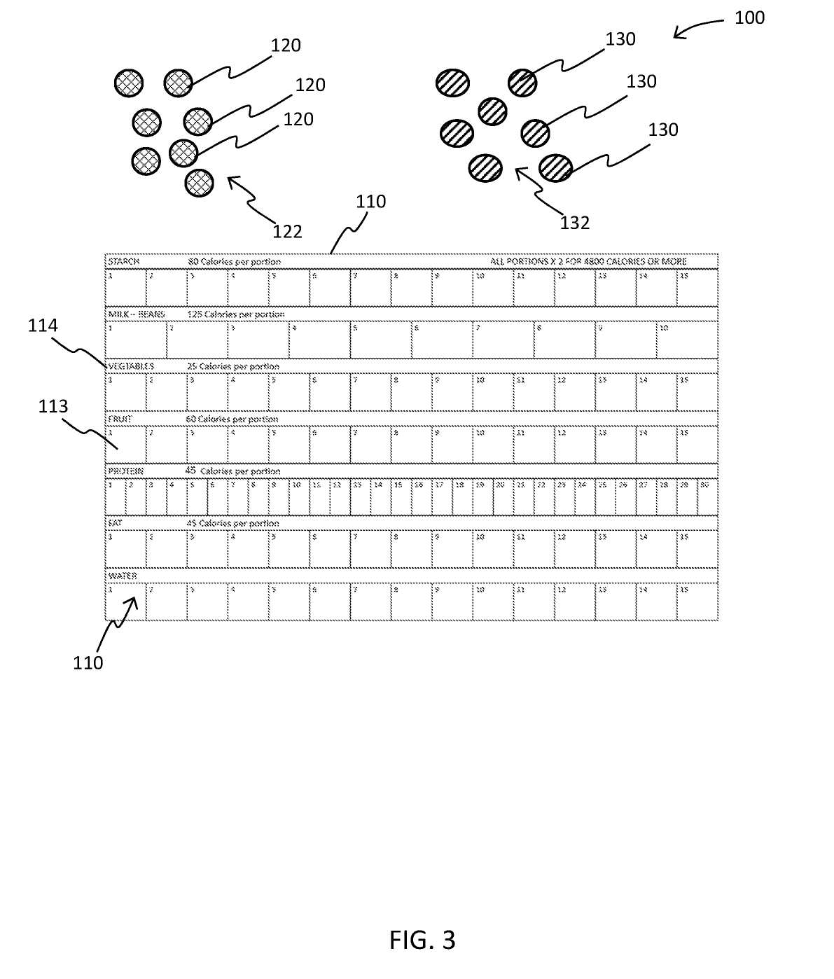

FIG. 3 shows a front perspective view of the system 100 of FIG. 1, according to an embodiment of the present disclosure. The plurality of first board-markers 120 may be configured for attachment to the food-board 110. As shown, the plurality of first board-markers 120 may include a first-format 122. Similarly, the plurality of second board-markers 130 may be configured for attachment to the food-board 110 and the plurality of second board-markers 130 may include a second-format 132. As shown here, the second-format 132 may be readily distinguishable from the first-format 122. In a preferred embodiment, the first-format 122 may be a first-color, and the second-format 132 may be a second-color. In this preferred embodiment, the first-color may be red and the second-color may be green.

A total number of the plurality of first board-markers 120 may be seven, and a total number of the plurality of second board-markers 130 may be seven. More or less may be used. In one example, the plurality of second board-markers 120 may be placed on the food-board 110 in each of the at least six food-rows 113 to represent a ‘stopping’ point for each food-group. In this example, for a user who is on a high protein diet a user may place one of the plurality of second board-markers 130 in the food-row 113 towards an end of the 113 to represent a high amount of protein is needed for their diet.

Further, the plurality of first board-markers 120 may be used to represent where the user is in terms of their meals for the day. The user may move the plurality of first board-markers 120 along the food-rows 113 as they eat from each food-group. In the example above, if the user consumes a high protein meal, they may move one of the plurality of first board-markers 120 along the food-row 113 that represents protein.

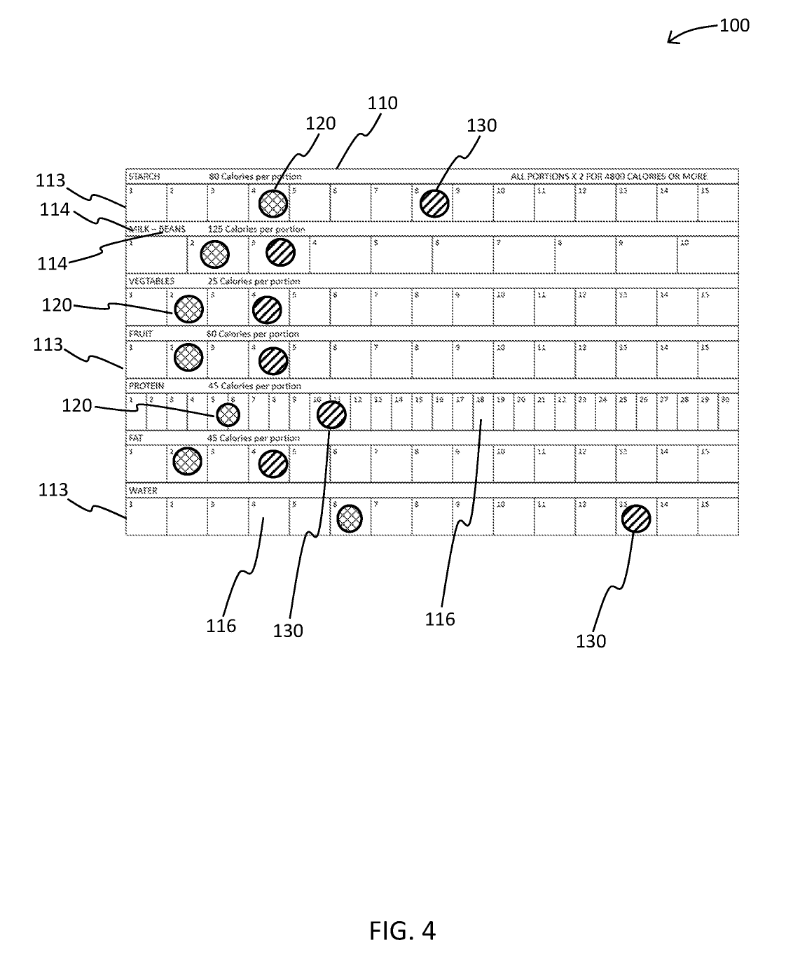

Referring now to FIG. 4 showing a front perspective view of the system 100 of FIG. 1, according to the embodiment of the present disclosure. Shown in this figure is a sample of the food-board 110 illustrating a “recommended balanced diet” according to the embodiment of the present disclosure. As above, the system 100 may include at least six food rows and one water row 113 (or five rows for vegans) may have at least one food group indicia 114 (or two food groups 114 if the calorie count is the same per portion), and a plurality of row cells 115. Shown here is ‘red stop’ markers 130 (in above example, the plurality of second board-markers 130) positioned for a 2000 calorie balanced diet, and a user's ‘green tracking’ markers 120 (in above example, the plurality of first second board-markers 120) showing the user has eaten half their daily goal, to lunch.

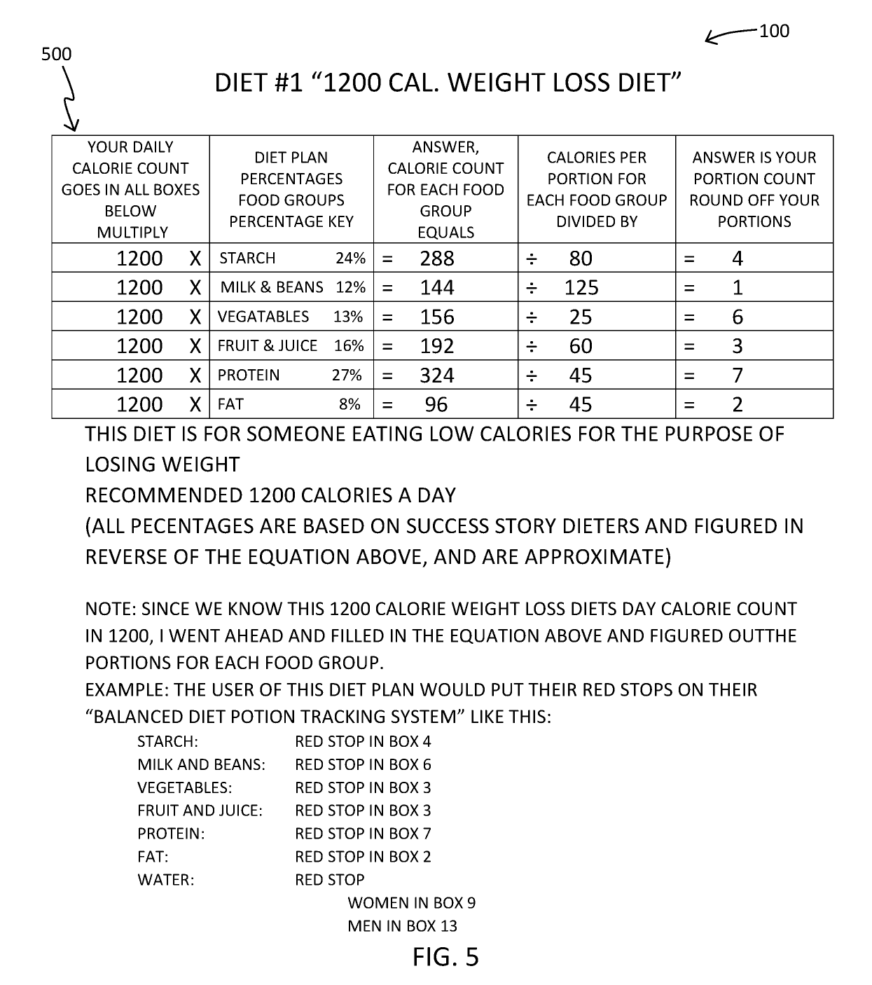

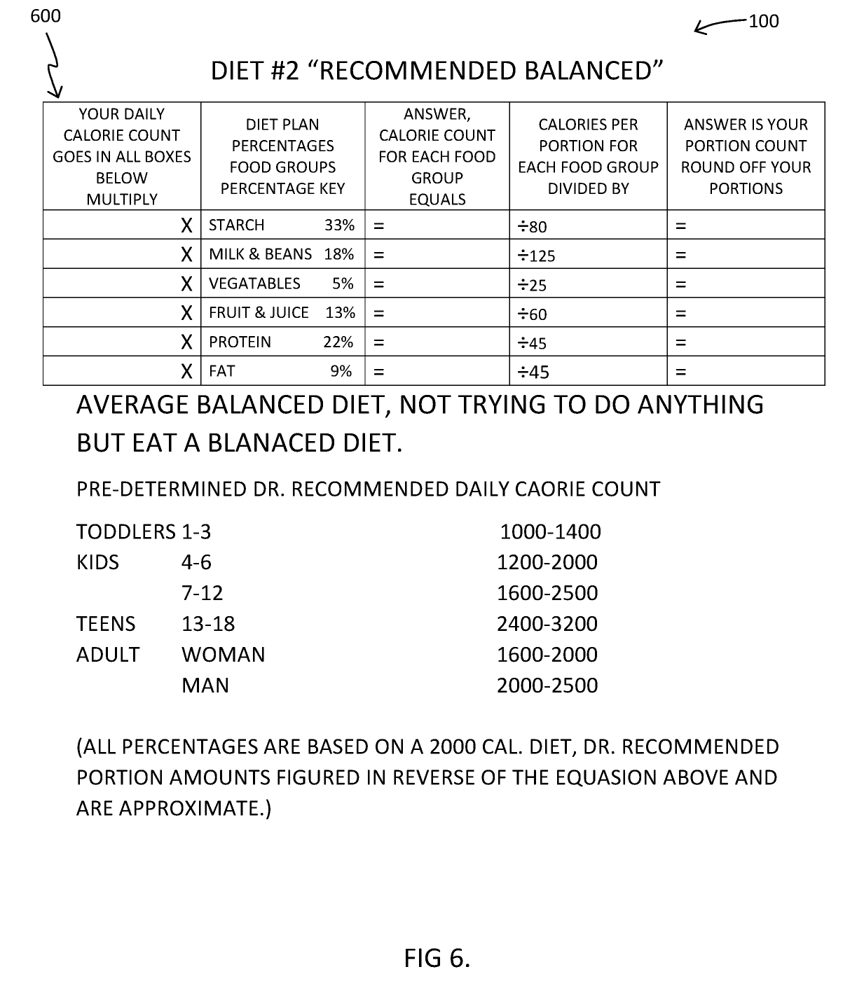

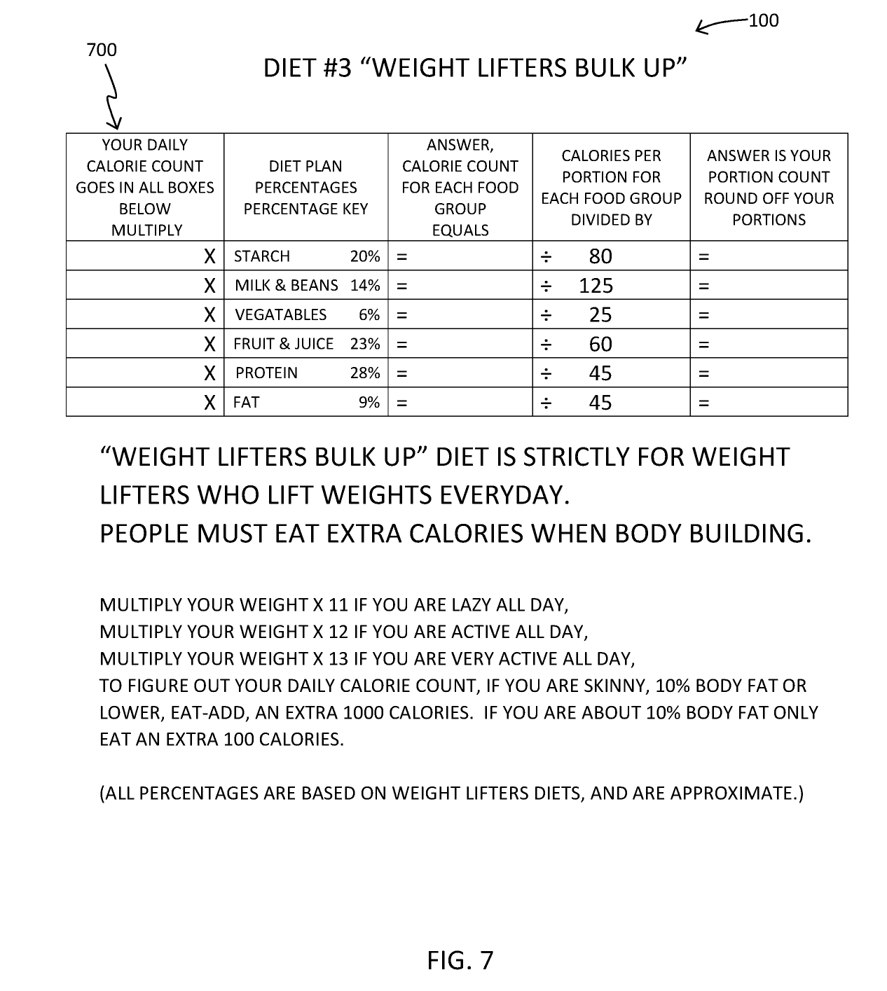

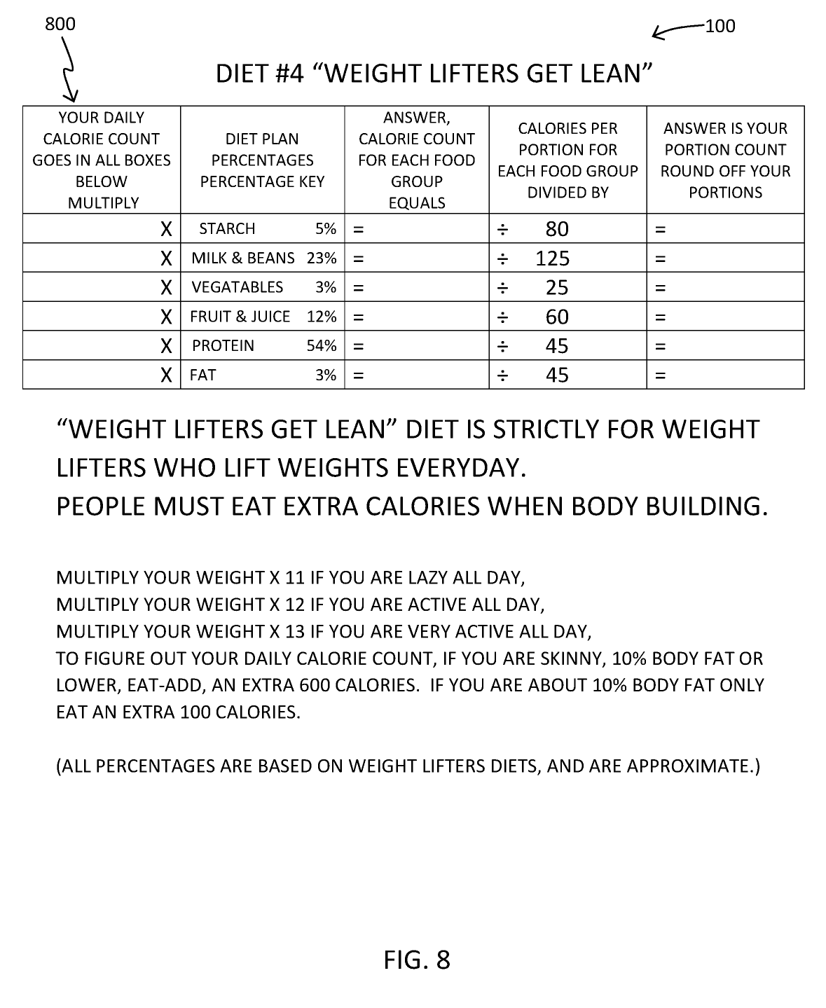

Referring now to FIG. 5 showing a front perspective view of the system 100 of FIG. 1, according to the embodiment of the present disclosure. As above, all “Balanced Diet Portion Tracking Systems” may be provided with instructions, and the instructions may include four different diet options. The four different diet options may include: one “1200 Calorie Weight Loss Diet” 500; one average “Dr. Recommend Balanced Diet” 600 (FIG. 6); one “Build Bulk Weight Lifters Diet” 700 (FIG. 7); and one “Get Lean Weight Lifters Diet” 800 (FIG. 8). These diets may be included in with the instructions to help people learn where to put their red stop markers on the “Balanced Diet Portion Tracking System”. Other systems may only cover a limited amount of calories, for small, medium and large people. Illustrated here is the “1200 Calorie Weight Loss Diet” 500.

As shown, this diet is someone eating low calories for the purpose of losing weight. All percentages calculated and shown in this diet may be based on success story dieters. Also shown is an example of how the user would put their “red stop” markers 120 on the food-board 110 of the system 100 (FIG. 3).

Referring now to FIG. 6 showing a front perspective view of the system 100 of FIG. 1, according to the embodiment of the present disclosure. Illustrated here is the “Dr Recommend Balanced Diet” 600 as mentioned above. As shown here, this diet illustrates an average balanced diet wherein the user is not trying to lose weight, but rather trying to eat an average balanced diet. All percentages may be based on a 2000 calorie diet.

Referring now to FIG. 7 showing a front perspective view of the system 100 of FIG. 1, according to the embodiment of the present disclosure. Illustrated here is the “Build Bulk Weight Lifters Diet” 700 as mentioned above. As shown here, this diet may be used for weight lifters who lift weights every day as the user may need to eat extra calories when body building. All percentages may be based on a weight lifters diet.

Referring now to FIG. 8 showing a front perspective view of the system 100 of FIG. 1, according to the embodiment of the present disclosure. Illustrated here is the “Get Lean Weight Lifters Diet” 800 as mentioned above. As shown here, this diet may also be used for weight lifters, as the diet above. This diet may also be based on weight lifters diets.

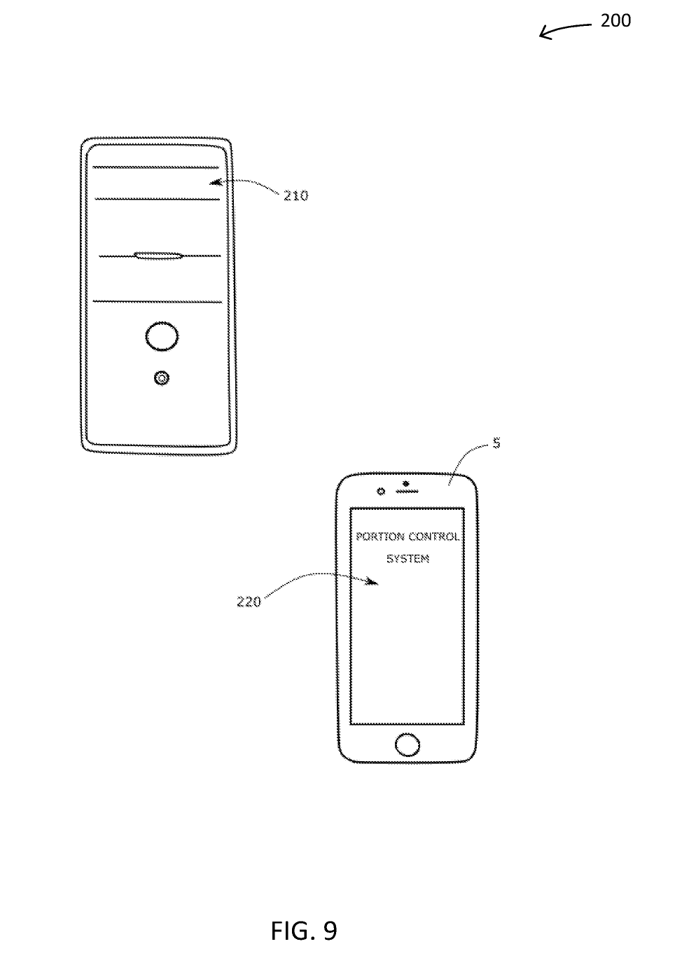

FIG. 9 shows a front perspective view of a system 200 of according to an alternative embodiment of the present disclosure. This system 200 may be for use on an electronic-device 5. As illustrated, the system 200 may include a server 210 and a software-application 220. This embodiment may be an electronic-replica of the system 100 as above.

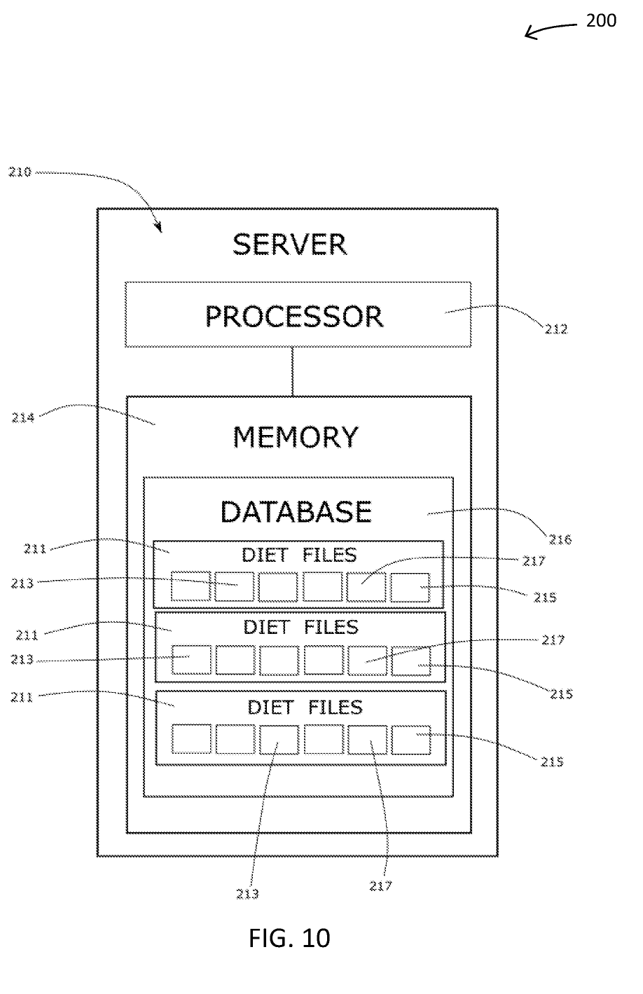

Referring now to FIG. 10 showing a front perspective view of a system 200 of according to an alternative embodiment of the present disclosure. The server 210 may include a server-processor 212 and a server-memory 214, and the server-memory 214 may have a server-database 216 including a plurality of food-portion control diet files 211. As illustrated, the plurality of food-portion control diet files 211 may each include a plurality of food-group files 213, a plurality of food-portion files 215 and at least one marker-position information file 217.

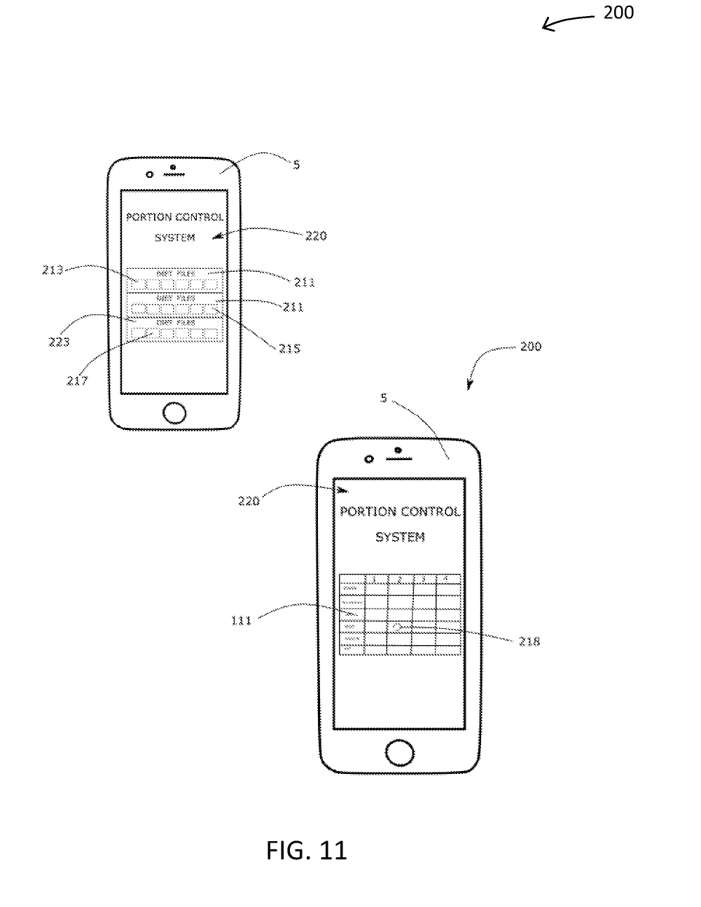

FIG. 11 shows a front perspective view of a system 200 of according to an alternative embodiment of the present disclosure. As illustrated, the software-application 220 may be downloadable to the electronic-device 5. The plurality of food-portion control diet files 211 may be configured for selection by a user via the software-application 220 on the electronic-device 5. Further, the plurality of food-portion control diet files 211 may be displayed as a list 223 via the software-application 220 on the electronic-device 5.

Upon selection of at least one of the plurality of food-portion control diet files 211, the plurality of food-group files 213 and the plurality of food-portion files 215 may be displayed as a food-table 111 on the software-application 220. Further, the at least one marker-position information file 217 may be displayed as a marker-position 218 on the food-table 111.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for system 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A system for tracking food portion control, the system comprising:
a food-board including a food-table being defined by at least two portion-columns and at least six food-rows, the at least six food-rows each having at least one food-group indicia and a plurality of row-cells, the at least one food-group indicia being different on each of the at least six food-rows, and wherein each of the plurality of row-cells includes at least one food-portion indicia corresponding to the at least one food-group indicia on each of the at least six food-rows;
a plurality of first board-markers configured for attachment to the food-board, the plurality of first board-markers including a first-format; and
a plurality of second board-markers configured for attachment to the food-board, the plurality of second board-markers including a second-format, and wherein the second-format is readily distinguishable from the first-format.

2. The system of claim 1, further comprising at least one liquid-row.

3. The system of claim 2, wherein the liquid-row includes at least one liquid-group indicia.

4. The system of claim 3, wherein the liquid-group indicia represent water.

5. The system of claim 1, wherein one of the at least six food-rows includes the at least one food-group indicia, and wherein the at least one food-group indicia represent starch.

6. The system of claim 5, wherein another one of the at least six food-rows includes the at least one food-group indicia, and wherein the at least one food-group indicia represent milk and beans.

7. The system of claim 6, wherein another one of the at least six food-rows includes the at least one food-group indicia, and wherein the at least one food-group indicia represent vegetables.

8. The system of claim 7, wherein another one of the at least six food-rows includes the at least one food-group indicia, and wherein the at least one food-group indicia represent fats.

9. The system of claim 8, wherein another one of the at least six food-rows includes the at least one food-group indicia, and wherein the at least one food-group indicia represent proteins.

10. The system of claim 9, wherein another one of the at least six food-rows includes the at least one food-group indicia, and wherein the at least one food-group indicia represent fruits.

11. The system of claim 1, wherein the food-board, the first board-markers, and the second board-markers are magnetic, and wherein the first board-markers and the second board-markers are configured for magnetic-attachment to the food-board.

12. The system of claim 1, wherein a total number of the first board-markers is seven.

13. The system of claim 12, wherein a total number of the second board-markers is seven.

14. The system of claim 1, wherein the first-format is a first-color, and the second-format is a second-color.

15. The system of claim 14, wherein the first-color is red.

16. The system of claim 14, wherein the second-color is green.

17. A system for tracking food portion control for use on an electronic-device, the system comprising:
a server including a server-processor and a server-memory, the server-memory having a server-database, the server-database including a plurality of food-portion control diet files, and wherein the plurality of food-portion control diet files each include a plurality of food-group files, a plurality of food-portion files and at least one marker-position information file; and
a software-application downloadable to the electronic-device; and
wherein the plurality of food-portion control diet files are configured for selection by a user via the software-application on the electronic-device.

18. The system of claim 17, wherein the plurality of food-portion control diet files are displayed as a list via the software-application on the electronic-device.

19. The system of claim 18, wherein upon selection of at least one of the plurality of food-portion control diet files, the plurality of food-group files and the plurality of food- portion files are displayed as a food-table on the software-application.

20. The system of claim 19, wherein the at least one marker-position information file is displayed as a marker-position on the food-table.
